(19) United States
(12) Patent Application Publication
(10) Pub. No.: US 2007/0178439 A1
(43) Pub. Date: Aug. 2, 2007

(54) Control of es cell self renewal and lineage specification, and medium therefor

(76) Inventors: Austin Gerard Smith, Edinburgh (GB); Qi-Long Ying, Edinburgh (GB); Jennifer Nichols, Edinburgh (GB)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/576,358

(22) PCT Filed: Oct. 15, 2004
(86) PCT No.: PCT/GB04/04401

§ 371 Date: Aug. 11, 2006

(30) Foreign Application Priority Data
Oct. 16, 2003 (GB) 0324270.8
Oct. 17, 2003 (GB) 0324378.9
Oct. 27, 2003 (GB) 03250073

(51) Int. Cl. C12Q 1/00; C12N 5/08; C12N 15/09

(52) U.S. Cl. 435/4; 435/455; 435/325; 435/366

(57) ABSTRACT

Self renewal of pluripotent cells in culture is promoted using a combination of an Id gene product and an activator of a gp130 downstream signalling pathway.

The present invention relates to media, culture conditions and methods of culturing pluripotent stem cells in order to promote stem cell self renewal and to prevent or control differentiation of the stem cells. The invention further provides methods for deriving, isolating and maintaining homogeneous preparations of pluripotent stem cells. The methods and compositions provided are suitable for culturing and isolating pluripotent stem cells such as embryonic stem (ES) cells, especially mammalian, including human, stem cells.

The establishment and maintenance of in vitro pluripotent stem cell cultures in the presence of medium containing serum and Leukaemia Inhibitory Factor (LIF) is well known (Smith et al. (1988) Nature 336: 688-90). Such methods have been used to maintain pluripotent embryonic stem (ES) cells from strains of permissive mice over many passages. Maintenance and self renewal of pluripotent stem cell cultures is further supported where the stem cells are cultured in the presence of feeder cells or extracts thereof, usually mouse fibroblast cells. Under such conditions it is possible to maintain human ES cells in a pluripotent state over many passages in culture.

A continuing problem in this field is that, despite intense efforts, it remains the case that pluripotent cultures of ES cells can be derived and maintained for extended periods only from a few species and even in those species not from all embryos. In some cases, pluripotent cells can be identified but can not then be maintained in culture for sufficient time to enable study of the cells or their genetic manipulation. This is particularly the case for rodent (other than some strains of mice) cells.

A further problem, until recently, was that ES cells that could indeed be maintained in a pluripotent state in culture over many passages could only be so maintained using medium that contained serum or serum extract, and hence was undefined, or used cell culture conditions that required the presence of other cells, such as the fibroblast feeder cells used to maintain human ES cells. However, where ES cells are intended to be subjected to subsequent controlled differentiation into desired cell types it is undesirable to utilise an undefined culture medium or to have heterologous cells present.

The serum typically used in culturing pluripotent stem cells is fetal calf (bovine) serum, which is known to contain a complex mixture of cytokines and other signalling molecules. In order to control differentiation pathways it is undesirable to introduce unknown cytokines to the culture medium as their influence on the eventual outcome of differentiation is unquantifiable, and could be potentially deleterious. Further, each serum batch is unique and introduces variation into culture protocols.

As a result, the ES cells obtained by culture in such complex media, and any differentiated progeny thereof, risk being contaminated by components of the media and/or by cells such as feeder cells that are required to maintain the ES cells. These factors mitigate against development of good manufacturing practices for therapeutic and other applications of ES cells and their progeny.

When deriving a differentiated cell population from an ES cell culture, it is desirable to be able to convert a high proportion of the ES cells into progeny of the same type—i.e. to maintain as homogeneous a population of cells as possible. However, in practice it is observed that, following differentiation, a cell population is obtained that contains a heterogenous mixture of cells. Hence, it is desirable to be able to carry out differentiation of an ES cell population in such manner or using such factors as to obtain a purer population of progeny.

In a prior application by the applicants, WO-A-03/095628, culturing pluripotent stem cells, such as ES cells, in serum-free media comprising agonists of the gp130 (e.g. LIF) and TGF-β superfamily (e.g. BMP4) signalling pathways is used to promote self renewal of the stem cells for multiple passages. In the presence of gp130 signalling, an agonist of the TGF-β superfamily signalling pathway surprisingly provided a self renewal stimulus rather than a pro-differentiation signal.

An object of the invention is to provide alternative, preferably improved, methods of culturing and culture media suitable for pluripotent stem cells, which are capable of supporting self-renewal of said stem cells in an undifferentiated state for many passages. A further object of the invention is to provide an alternative culturing system that permits maintenance of a pluripotent stem cell culture in vitro until differentiation of the cells is induced in a controlled manner. A still further object of the invention is to provide methods and compositions that enhance the derivation and isolation of pluripotent stem cells and facilitate their derivation and isolation from organisms refractory to ES cell isolation or from which pluripotent stem cells have not yet been isolated.

According to the present invention, provision of Id proteins in a pluripotent cell in the presence of gp130 signalling suppresses differentiation and promotes self-renewal.

In the present invention, pluripotent stem cells, such as ES cells, are cultured in serum-free media comprising agonists of the gp130 (e.g. LIF) signalling pathway coincident with Id gene expression, such as via (i) direct activation of the Smad pathway to express Id genes, or expression of an Id gene, or (ii) presence in the cells of Id gene product or an equivalent signal. Self renewal of the stem cells for multiple passages is thereby promoted. Hence, in the presence of gp130 signalling, Id gene activity in the pluripotent cells provides a self renewal stimulus.

The present invention hence provides the use of an Id gene product in promoting self-renewal of pluripotent cells in culture. Following the invention, Id gene products are purposively provided in cells and/or Id genes or equivalent are purposively activated. With coincident gp130 signalling, especially using a cytokine such as LIF, self renewal of pluripotent cells has been obtained.

It is known from an earlier application by the inventors to promote self renewal using LIF and activation of signalling downstream of a receptor of the TGF-β superfamily. In the present invention, Id gene activity in combination with LIF results in self renewal of ES cells. Hence the invention provides additional means of providing that self renewal signal, namely through a combination of
(i) an Id protein or an agent that increases Id protein activity, said agent being other than an activator of a signalling pathway downstream from a receptor of the TGF-β superfamily; and
(ii) an activator of a gp130 downstream signalling pathway,

Reference to Id protein includes fusions of Id proteins with other proteins, e.g. translocation domains, and compositions comprising Id proteins as described below. The agent of (i) is suitably an extrinsic factor that induces Id gene expression and/or induces Id protein activity without acting through a receptor of the TGF-beta superfamily. Examples include fibronectin, agonists of the fibronectin receptor, activators of integrin signalling, nanog, and homologes of all of the aforementioned that induce Id gene expression or Id protein activity. The methods of the invention are suitable for culture of pluripotent stem cells, especially embryonic stem cells.

In examples below, we have induced expression of an Id gene to promote self renewal. In another embodiment of the invention, also described below, we have genetically manipulated a pluripotent cell so that it expresses an Id gene, for example by introducing into a pluripotent cell a vector comprising an Id gene. Precise control can be achieved using an inducible promoter in the vector. This type of genetic modification is acceptable where the cells are used for drug screens, but may not be so where the cells or progeny are to be used therapeutically—in which case use of extrinsic factors to promote self renewal is referred.

In specific methods described below in more detail, a method of promoting self-renewal of pluripotent cells in culture comprises (i) expressing an Id gene or inducing expression of an Id gene, and (ii) activating gp130 downstream signalling. The Id gene can conveniently be expressed episomally.

A further aspect of the invention provides the use of a combination of:—
(a) an activator of Id gene expression and/or Id protein activity which results in expression of an Id gene; and
(b) an activator of a gp130 downstream signalling pathway, in promoting self-renewal of pluripotent cells in culture.

An alternative aspect of the invention provides the use of a combination of:—
(a) an Id gene product; and
(b) an activator of a gp130 downstream signalling pathway, in promoting self-renewal of pluripotent cells in culture.

In a specific embodiment of the invention, described in more detail below LIF is included in medium in which an ES cell constitutively expressing Id1 is cultured. Self renewal was enhanced, demonstrating the synergy of LIF and the expressed Id gene.

An advantage of the invention is the direct provision in the cells of an Id gene product, in promotion of self renewal. With direct induction of self renewal comes greater control of self renewal, with less side effects attributable for example to activation of pathways several stages away from the agents of the self renewal mechanism.

Reference to Id genes is intended to encompass the genes as so defined in the literature, e.g. Id1, Id2, Id3, and Id4, and is also intended to encompass mimics thereof, including functional fragments and derivatives, which exhibit the property of Id gene products, namely that of inhibiting the transcriptional activity of bHLH factors such as myoD and mash1. Specific mouse, rat, canine and human Id protein sequences are set out in SEQ ID NO:s 1-4. Other specific Id protein sequences are obtainable via publicly available sequence database. Id gene activity can suitably be mimicked by preventing or reducing expression or activity of a bHLH gene or preventing or reducing expression or activity of an E protein. This may be achieved using gene knock-out or inhibitory RNA strategies or by eliminating extrinsic inducing factors. An antisense RNA may be used in one RNA targeting method, or an siRNA based approach can be used.

A signal equivalent to increased Id protein activity may be provided by (i) an inhibitor of a bHLH gene, (ii) an inhibitor of myoD, (iii) an inhibitor of mash1, (iv) increased hes gene activity, (v) increased hes protein activity, and (vi) combinations of one or more of all of the above.

In the art, a factor such as BMP is used to activate one or more signalling pathways downstream from a receptor of the TGF-β superfamily. The present invention differs therefrom in that it relies on direct provision of Id gene activity in the cells, e.g. through a vector expressing an Id gene, or it relies on activation of Id gene expression and/or Id protein activity, other than via a receptor of the TGF-β superfamily or directly mimic the effects of such activation. The invention can be more targeted than the art and enable greater precision in maintaining a self renewing phenotype than hitherto.

Activation of one or more gp130 downstream signalling pathways can be achieved by use of a cytokine acting through gp130, for example a cytokine or other agonist of the LIF receptor.

Cytokines capable of acting through gp130, and thus of activating gp130 signal transduction, include LIF, CNTF, cardiotrophin, oncostatin M, IL-6 plus sIL-6 receptor and hyper 11-6. Suitable cytokines include mimetics, fusion proteins or chimaeras that can bind to and/or activate signalling though gp130. The role of cytokines acting through gp130 in the presence of serum is well established, but the capacity of those cytokines to sustain undifferentiated cells in the absence of serum is limited.

The present invention therefore provides, in one embodiment, alternative and/or improved culture of ES cells in medium that is free of serum, serum extract, feeder cells and feeder cell extract. When using the LIF and direct Id gene expression and/or Id protein activity-activating medium of a specific embodiment of the present invention extended passaging of ES cells is possible.

Another advantage of the present culture system is that differentiation of ES cells is reduced compared to culture in the presence of serum. This is significant because often the most pluripotent ES cells tend to differentiate considerably in serum, making their manipulation and expansion problematic. The results show that the culture conditions of the present invention enable ES cells to self-renew in the absence of serum.

Embryonic stem cells have been reported from a number of mammalian sources including mouse (Bradley et al (1984) Nature 309: 255-56), American mink (Mol Reprod Dev (1992) December; 33(4):418-31), pig and sheep (J Reprod Fertil Suppl (1991); 43:255-60), hamster (Dev Biol (1988) May; 127(1):224-7) and cow (Roux Arch Dev Biol (1992); 201: 134-141). It will be appreciated that the methods and compositions of the present invention are suitable for adaptation to culturing of other mammalian pluripotent cell cultures, including primate, especially human, rodent, especially mouse and rat, and avian ES cells.

Specifically, with regard to human ES cells, it is known that human ES cells respond to LIF and therefore the medium and methods of the invention, in which a self-renewal stimulus is obtained in response to a combination of LIF and Id proteins, are of application to human ES cells.

Suitable cell densities for the methods of the invention will vary according to the pluripotent stem cells being used and the natures of any desired progeny. Good results have been obtained by culturing embryonic stem cells in monolayer culture, dissociating the embryonic stem cells and subsequently culturing the embryonic stem cells in monolayer culture on a culture surface at a density of from 0.2-2.5×10⁴ cells per cm², more particularly at a density of from 0.5-1.5×10⁴ per cm². The cells proliferate as adherent monolayers and are observed to have a doubling time comparable to ES cells grown in serum-containing media together with LIF.

Typical surfaces for culture of ES cells and their progeny according to the invention are culture surfaces recognized in this field as useful for cell culture, and these include surfaces of plastics, metal, composites, though commonly a surface such as a plastic tissue culture plate, widely commercially available, is used. Such plates are often a few centimetres in diameter. For scale up, this type of plate can be used at much larger diameters and many repeats plate units used.

It is further common for the culture surface to comprise a cell adhesion protein, usually coated onto the surface. Receptors or other molecules on the cells bind to the protein or other cell culture substrate and this promotes adhesion to the surface and it is suggested promotes growth. Gelatin coated plates are commonly available and are suitable for the invention, and other proteins may also be used.

In an embodiment of the present invention, including an agent that suppresses differentiation, such as an inhibitor of the FGF receptor or of MEK/Erk signalling in the culture medium for at least part of the culturing period is found to suppress the tendency of ES cells to differentiate. In one embodiment, the ES cells are cultured in defined serum-free media comprising LIF and an FGF receptor inhibitor for a specified period before the FGF receptor inhibitor is removed and replaced by a direct activator of Smad signalling. FGF receptor inhibitors are used especially for cells other than human cells, and examples include the compounds SU5402 and PD173074. Alternatively, a competitive inhibitor of the FGF receptor can be used, suitably a soluble form of the receptor. Suitable MEK/Erk inhibitors include PD98059, U0126 and PD184352.

In an alternative embodiment, it is an option not to remove the FGF receptor or MEK/Erk inhibitor. Hence, the inhibitor is present in the culture medium for an extended period, either in the presence or absence of inducers of Id proteins. ES cells can thus be grown in culture for at least 20 passages in N2B27 medium in the presence of LIF and an FGF inhibitor. If the inhibitor is not removed from the medium, it is preferred that it is a specific inhibitor and has little or no activity on other receptors.

A second aspect of the invention provides a method of culture of ES cells so as to promote ES cell self renewal, comprising maintaining the ES cells in medium containing:—
(1) (a) an activator of an intracellular signalling pathway, other than one acting through a receptor of the TGF-β superfamily, which results in expression of an Id gene, or (b) an Id gene product; and
(2) an activator of a gp130 downstream signalling pathway.

Methods of the invention can be used for stimulating self-renewal of ES cells in medium which is free of serum and free of serum extract, which cells have previously been passaged in the presence of serum or serum extract. Preferably, such methods are also carried out in the absence of feeder cells and/or feeder cell extracts. For example, culture of ES cells can be carried out comprising the steps of:—
maintaining the ES cells in a pluripotent state in culture, optionally on feeders, in the presence of a cytokine acting though gp130 and serum or an extract of serum;
passaging the ES cells at least once;
withdrawing the serum or the serum extract from the medium and withdrawing the feeders (if present), so that the medium is free of feeders, serum and serum extract; and
subsequently maintaining ES cells in a pluripotent state in the presence of a direct activator or effector of Id gene expression and/or Id protein activity (other than one acting through a TGF-β receptor) and an activator of a gp130 downstream signalling pathway.

At around the time that the serum or extract of serum is withdrawn from the medium, it is an option to add to the medium an agent that suppresses differentiation, for example, an FGF-receptor inhibitor. It is an option for the inhibitor of differentiation to be withdrawn at the same time as or subsequent to maintenance of the cells in the presence of an Id protein. The serum or extract can be withdrawn at the same time as or before or after the feeder cells or extract is withdrawn.

The present invention also provides a method of obtaining a transfected population of ES cells, comprising:—
transfecting ES cells with a construct encoding a selectable marker;
plating the ES cells;
culturing the ES cells in the presence of a direct activator or effector of Id gene expression and/or Id protein activity; and an activator of gp130 downstream signalling pathways; and
selecting for cells that express the selectable marker.

The selectable marker may encode antibiotic resistance, a cell surface marker or another selectable marker as described e.g. in EP-A-0695351, and preferably comprises a nucleotide sequence encoding the selectable marker operatively linked to a promoter which preferentially expresses the selectable marker in desired cells.

In a further embodiment, the present invention provides a method of culture of ES cells, comprising the steps of transferring an individual ES cell to a culture vessel, such as an individual well on a plate, and culturing the ES cell in the presence of a direct activator or effector of a Smad signalling pathway and an activator of gp130 downstream signalling pathways, so as to obtain a clonal population of ES cells, all of which are progeny of a single ES cell.

Once a stable, homogenous culture of ES cells is obtained, the culture conditions can be altered to direct differentiation of the cells into one or more cell types selected from ectodermal, mesodermal or endodermal cell fates. Addition of, or withdrawal of cytokines and signalling factors, can enable the derivation of specific differentiated cell populations at high efficiency. Differentiation of an ES cell towards a non-neuroectodermal fate may be achieved by maintaining the ES cell in the presence of a cytokine acting through gp130 and a direct activator or effector of a Smad signalling pathway and then withdrawing the cytokine whilst maintaining the direct activator or effector of a Smad signalling pathway and/or adding a further signalling molecule capable of directing differentiation. The methods described above all optionally includes the step of obtaining and/or isolating a differentiated cell which is the product of the process.

For example, exposure to BMP4 in the absence of LIF leads to induction of mesoderm and endoderm cell types. Withdrawal of agonists of the gp130 and TGF-β signalling pathways and/or blockade of both pathways leads to induction of a neurectodermal phenotype. Alternatively, other signalling factors can be added to the culture conditions to direct other differentiation pathways—for example, activin, sonic hedgehog (shh), Wnts and FGFs.

In use, towards the end of ES cell culture, it is desirable to remove the Smad signal at least one passage before differentiation is initiated, in order to ensure that the signal declines and there is no legacy of the signal during subsequent differentiation. In one embodiment, an FGF receptor antagonist is added to the cultures for one to two passages whilst removing the direct activator or effector of Id gene expression and/or Id protein activity.

Further aspects of the invention provide for cell culture media for self-renewal of ES cells. One such medium comprises:—
basal medium;
a direct activator or effector of Id gene expression and/or Id protein activity;
an activator of gp130 downstream signalling pathways; and
an iron-transporter;
   wherein the medium is optionally free of serum and serum extract.

Preferred medium for human pluripotent stem cells comprises a direct activator or effector of Id gene expression and/or Id protein activity; an activator of gp130 downstream signaling pathways and an agonist of the FGF receptor. Preferred medium for pluripotent stem cells other than human stem cells comprises a direct activator or effector of Id gene expression and/or Id protein activity; an activator of gp 130 downstream signaling pathways and an inhibitor ES cell differentiation.

Basal medium is medium that supplies essential sources of carbon and/or vitamins and/or minerals for the ES cells. The basal medium is generally free of protein and incapable on its own of supporting self-renewal of ES cells. The iron transporter provides a source of iron or provides ability to take up iron from the culture medium. Suitable iron transporters include transferrin and apotransferrin.

It is preferred that the medium further comprises one or more of insulin or insulin-like growth factor and albumin (preferably recombinant), and is free of feeder cells and feeder cell extract.

A particular medium of the invention comprises LIF, BMP, insulin, albumin and transferrin, with or without additional basal medium.

The invention also provides cell culture Media comprising:—
a direct activator or effector of Id gene expression and/or Id protein activity; and
a cytokine acting through gp130.

The culture medium is optionally supplemented with an inhibitor of differentiation of ES cells as described above, or, when differentiation is desired, signalling factors that direct differentiation of ES cells toward a specific phenotype.

It is preferred that the medium is free of serum or serum extract. Most preferably, the medium is fully defined.

In a preferred embodiment of the invention the culture medium comprises the gp130 receptor binding cytokine, LIF, at a concentration of between 10 U/ml and 1000 U/ml, more preferably between 50 U/ml and 500 U/ml, even more preferably in the region of 100 U/ml.

A specific human pluripotent stem cell medium comprises (a) LIF, (b) a BMP and (c) FGF. A specific medium for non-human pluripotent stem cells comprises (a) LIF, (b) a BMP and (c) an inhibitor of FGF. Substitutions of media components can be made as described herein.

The invention further provides a method of deriving a pluripotent cell from a blastocyst, comprising:—
(1) obtaining a blastocyst;
(2) culturing the blastocyst in the presence of an activator of gp130 downstream signalling, to obtain an inner cell mass;
(3) dissociating the inner cell mass;
(4) isolating a cell or cells from the dissociated inner cell mass; and
(5) culturing the isolated cell or cells in the presence of an activator of gp130 downstream signalling and an activator of Id gene expression or a product of Id gene expression.

Preferably, the method comprises culturing the blastocyst in LIF, more preferably for a period of from 2 to 4 days.

The isolated cell or cells are preferably cultured in serum free medium. Typically, the cells are replated as clumps. In examples below, we have obtained good results using a combination of LIF and an agonist of the BMP receptor.

The blastocyst is also preferably cultured in serum free medium, optionally in the absence of an agonist of the BMP receptor.

Still further provided in the invention is a vector, comprising an Id gene operatively linked to a promoter.

The promoter is suitably an inducible promoter, providing for control of expression using extinsic factor. It can be an episomal vector, e.g. as described in examples below.

A further culture medium of the invention is one comprising an agent which induces Id protein expression, other than an agent acting through a receptor of the TGF-β superfamily of receptors. Examples include fibronectin, agonists of the fibronectin receptor, activators of integrin signalling, nanog, and homologes of all of the aforementioned that induce Id gene expression or Id protein activity.

The medium may comprise an Id protein, e.g an Id protein linked to a translocation domain, to facilitate translocation of the Id protein across the cell membrane of a pluripotent cell.

“Translocation domain” means a domain or fragment of a protein which effects transport of itself and/or other proteins and substances across a membrane or lipid bilayer and encompasses native domains and fragments, variants and derivatives that retain this binding function. The latter membrane may be that of an endosome where translocation will occur during the process of receptor-mediated endocytosis. Translocation domains can frequently be identified by the property of being able to form measurable pores in lipid membranes at low pH (Shone et al. (1987) Eur J. Biochem. 167, 175-180 describes a suitable test). The latter property of translocation domains may thus be used to identify other protein domains which could function as the translocation domain within the construct of the invention. Examples of translocation domains derived from bacterial neurotoxins are as follows:—
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

Other suitable translocation domains are TAT (e.g. from HIV-1) and penetratin, short sequences of amino acids that internalize covalently linked peptides and convey them, or enable them to be conveyed, to the nucleus. Further suitable domains, referred to as protein transduction domains, such as VP22, derivatives of antennapedia and others, are described in Wadia et al., 2002. These domains can be linked to an Id protein chemically, e.g. via thiol functional groups or a fusion can be expressed comprising the Id protein and the domain. Specific domains are set out in SEQ ID NO:s 5-6 and specific fusion proteins comprising an Id protein and a protein transducing domain are set out in SEQ ID NO:s 7-9. The linked molecules, the fusions and compositions comprising the same from another aspect of the invention. These can be used e.g. as additives to culture medium as an alternative to transfecting cells with an Id gene.

“Translocation” in relation to translocation domain, means the internalization events which occur after binding to the cell surface. These events lead to the transport of substances into the cytosol of cells.

A composition for delivery of a factor to an ES cell therefore comprises:—
the factor, and
a translocation domain that translocates the factor into the ES cell, suitably
a H_{N} domain of a clostridial toxin.

The translocation domain can also be selected from (1) a H_{N} domain of a diphtheria toxin, (2) a fragment or derivative of (1) that substantially retains the translocating activity of the H_{N} domain of a diphtheria toxin, (3) a fusogenic peptide, (4) a membrane disrupting peptide, and (5) translocating fragments and derivatives of (3) and (4).

Yet further provided in the invention is use of an agent that increases Id protein activity in a pluripotent cell, in promoting self-renewal of the pluripotent cell.

The agent is suitably as described elsewhere herein, and may be one that increases the amount of Id protein in the cell or enhances the activity of Id protein in the cell.

It will be appreciated by a person of skill in the art that activation of signalling pathways downstream from a receptor of the TGF-β superfamily can be effected by either upstream agonists of the TGF-β receptor (e.g. receptor ligands), constitutively active receptors, or activated downstream components of the signalling pathway, for example the SMAD signal transduction molecules. Likewise upstream effectors (eg. cytokines) and downstream effectors (eg. Stats) of the gp130 signal transduction pathway are capable of activating this pathway also. Thus, embodiments of the invention which refer to activation of signalling downstream of a TGF-β receptor, for example the methods of ES cell derivation, embrace all compositions comprising molecules capable of activating TGF-β receptor superfamily signalling pathways, preferably acting through the BMP receptor, in order to promote self renewal of pluripotent stem cells. Suitable ligands for the BMP receptor include BMPs and GDF.

It is further preferred, according to the invention, that culture of cells is carried out in an adherent culture, and in examples of the invention it has been found that following maintenance of cells in a pluripotent state, differentiation can be induced with a high degree of uniformity and with high cell viability. Adherent cultures may be promoted by the inclusion of a cell adhesion protein, and in specific examples of the invention gelatin has been used as a coating for the culture substrate.

It is also preferred to culture pluripotent cells according to the invention in monolayer culture, though it is optional for cells to be grown in suspension culture or as pre-cell aggregates; cells can also be grown on beads or on other suitable scaffolds such as membranes or other 3-dimensional structures.

A further component of medium for culture of pluripotent cells according to the invention, and which is preferred to be present, is a factor promoting survival and/or metabolism of the cells. In a specific embodiment of the invention, cells are cultured in the presence of insulin. An alternative factor is insulin-like growth factor and other such survival and/or metabolism promoting factors may alternatively be used.

Culture medium used in the examples of the invention preferably also comprises serum albumin. This can be used in purified or recombinant form, and if in a recombinant form this has the advantage of absence of potential contaminating factors, cytokines etc. The culture medium does not need to contain serum albumin and this component can be omitted or replaced by another bulk protein or by a synthetic polymer (polyvinyl alcohol) as described by Wiles et al.

A particularly preferred medium of the invention is one that is fully defined. This medium does not contain any components which are undefined, that is to say components whose content is unknown or which may contain undefined or varying factors that are unspecified. An advantage of using a fully defined medium is that efficient and consistent protocols for culture and subsequent manipulation of pluripotent cells can be derived. Further, it is found that maintenance of cells in a pluripotent state is achievable with higher efficiency and greater predictability and that when differentiation is induced in cells cultured using a defined medium the response to the differentiation signal is more homogenous then when undefined medium is used.

A medium according to the present invention may be used for culture of pluripotent stem cells from any adult tissue.

Methods of the invention also include a method of obtaining a differentiated cell comprising culturing a pluripotent cell as described and allowing or causing the cell to differentiate, wherein the cell contains a selectable marker which is capable of differential expression in the desired differentiated cell compared with other cell-types, including pluripotent stem cells, whereby differential expression of the selectable marker results in preferential isolation and/or survival and/or division of the desired differentiated cells.

The differentiated cell can be a tissue stem or progenitor cell, and may be a terminally differentiated cell.

The present invention also provides a method of isolating a pluripotent stem cell or an EG or EC cell comprising culturing cells or tissue from an embryo, or somatic cells from a fetus or adult in medium containing:—
a cytokine acting through gp130; and
an Id protein or a direct activator or effector of Id gene expression and/or Id protein activity; and/or
an inhibitor of a FGF receptor or of MEK/Erk.

Preferably, the medium is a fully defined medium.

Generally also, the invention extends to a cell obtained by following any of the methods of the invention described herein. Cells of the invention can be used in assays for drug discovery. Cells of the invention may also be used for cell therapy, and thus a method of the invention comprises using a combination of gp130 signalling and Id protein activity and/or expression of the invention to derive and/or maintain pluripotent cells, deriving cells for cell therapy therefrom and using those cells in cell therapy.

A method of reprogramming cells is provided by the invention, yielding pluripotent cells from non-pluripotent cells. Hence a method of obtaining a pluripotent cell comprises expressing an Id gene or inducing expression of an Id gene in a cell, or culturing a cell in medium containing an Id protein, and activating gp130 downstream signalling in the cell, wherein the cell is obtained from somatic cells or tissue of a fetus or adult. The pluripotent cell obtained is preferably characterised by being positive for Rex1, Oct4 and nanog.

An assay is provided by the invention, for a factor with activity that substitutes for an Id protein, said assay comprising:—
(1) culturing a cell in the presence of Id protein activity and gp130 downstream signaling, thereby maintaining the cell in a pluripotent state;
(2) removing or reducing the Id protein activity;
(3) introducing the factor into the cell; and
(4) determining whether the cell remains pluripotent or differentiates.

Culturing the cell in the presence of Id protein activity in (1) suitably comprises (a) expressing an Id gene, (b) inducing expression of an Id gene or (b) adding an Id protein to medium in which the cell is cultured, and introducing the factor into the cell suitably comprises (a) expressing the factor, or (b) adding the factor to medium in which the cell is cultured. A further aspect of the invention extends to a factor thereby obtained.

Id genes are prominent targets of BMP/Smad signalling in undifferentiated ES cells. Ids are negative helix loop helix factors that sequester E proteins to prevent the transcriptional activity of bHLH factors such as myoD and mash1 (Jen et al., 1992; Lyden et al., 1999) and are a candidate for being negative regulators of haematopoiesis (Nogueira et al, 200). They can also interact with and inhibit Pax and Ets transcription factors (Norton, 2000). In a specific embodiment of the invention, ES cells transfected with Ids self-renew in serum-free culture on addition of LIF alone, establishing that a critical contribution of BMP/Smad is to induce Id expression.

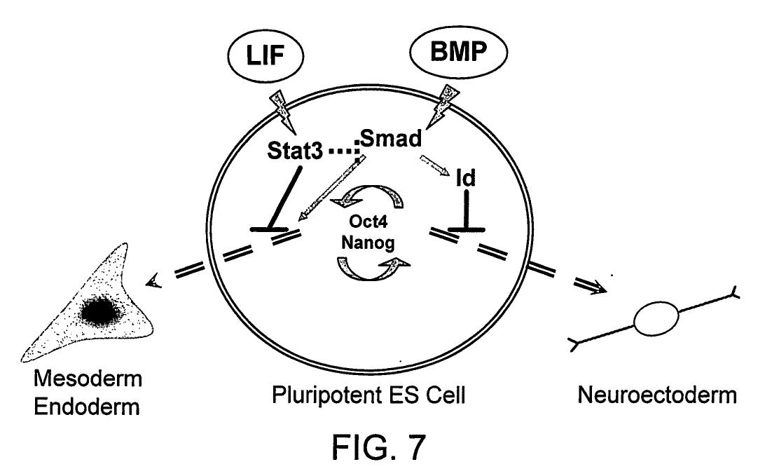

On LIF withdrawal, Id expressing ES cells readily differentiate but do not give rise to neural precursors. Thus Id proteins act in a lineage-specific manner, suppressing neural determination with little or no effect on mesoderm or primitive endoderm commitment. Ids therefore contribute to self-renewal by complementing the blockade of other lineages by STAT3 (FIG. 7). At least part of Id function may be to block the action of prematurely expressed pro-neural factors. Ids may thus act to insulate the stem cell from functional consequences of lineage priming (Hu et al., 1997).

LIF/STAT3 and BMP/Smad hence act in combination to sustain ES cell self-renewal. These two pathways also mediate ventralisation of the Xenopus embryo (Nishinakamura et al., 1999). In that case, each appears to be sufficient independent of activity of the other, with no evidence of cross-regulation between STAT3 and Smad1.

The homoedomain protein Nanog can bypass the requirement for activation of STAT3 in serum-containing medium (Chambers et al., 2003). Nanog can also be used to replace the requirement for BMP/serum stimulation, at least in part by conferring constitutive expression of Id.

There now follows illustrative examples of the invention, accompanied by drawings in which:—

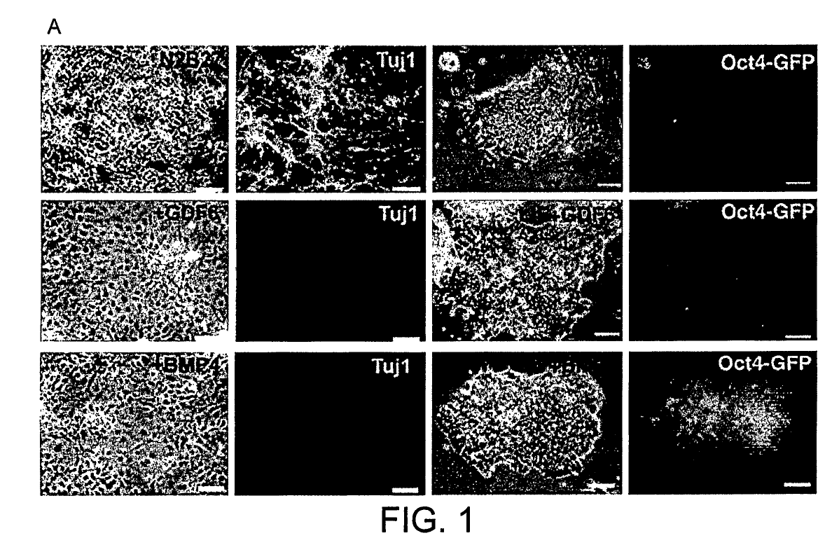

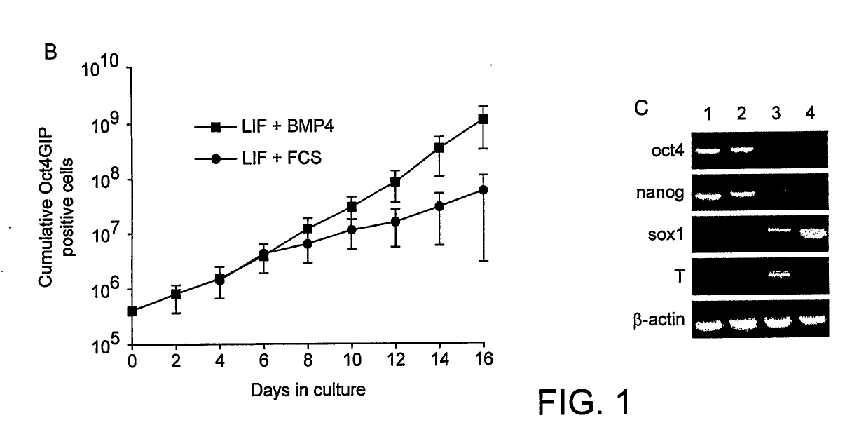

FIG. 1 shows LIF plus BMP sustain ES cell self-renewal in serum-free medium;

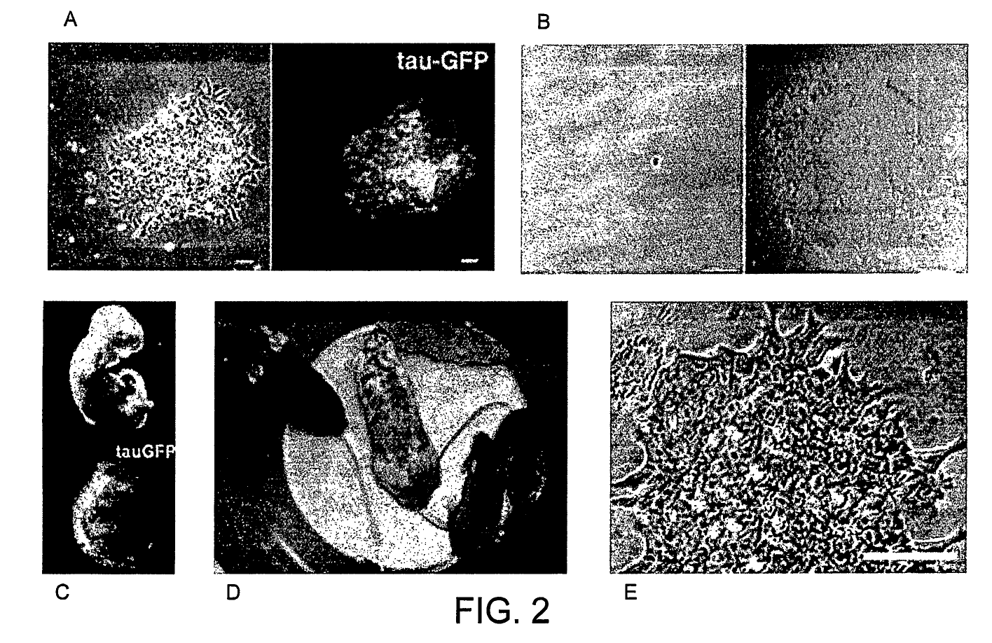

FIG. 2 shows clonogenicity, potency and derivation of ES cells in N2B27 with LIF plus BMP;

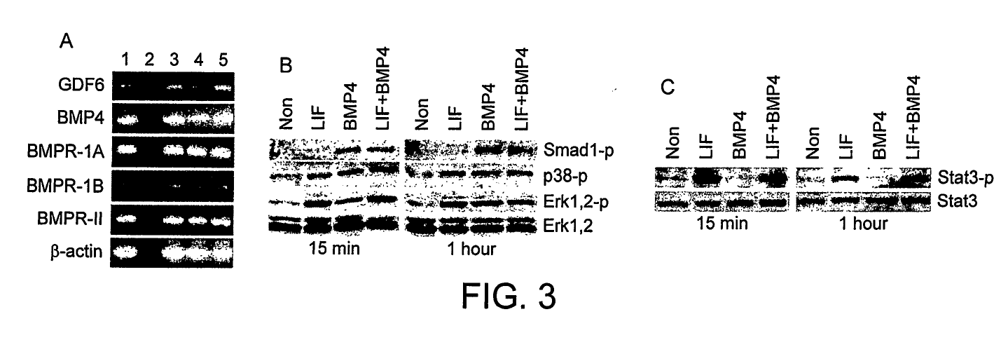

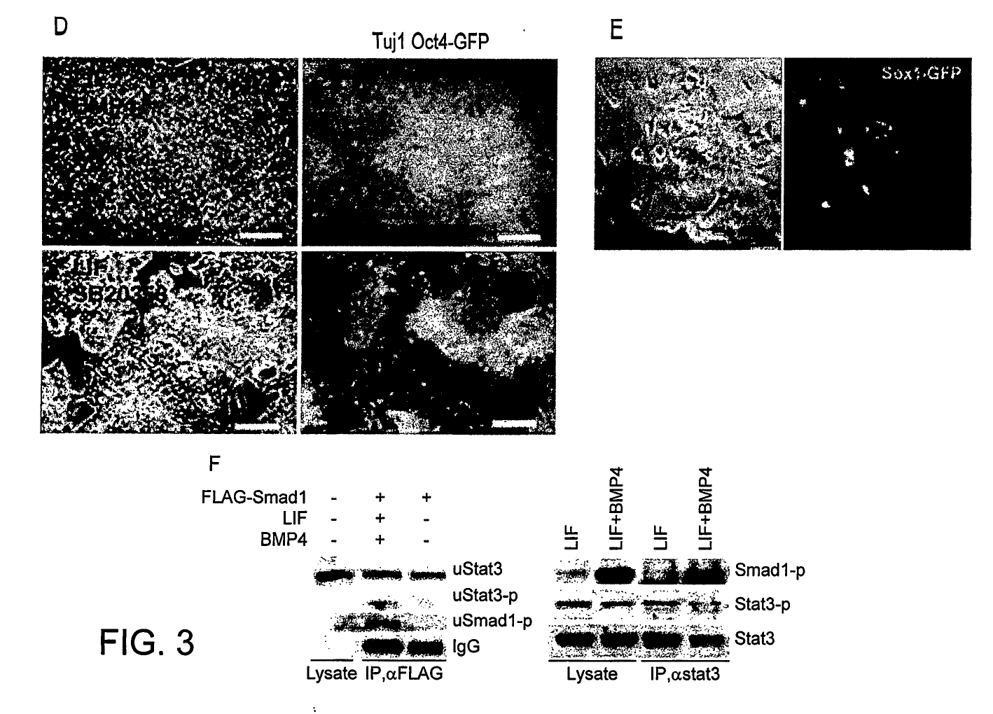

FIG. 3 shows BMP signalling in ES cells;

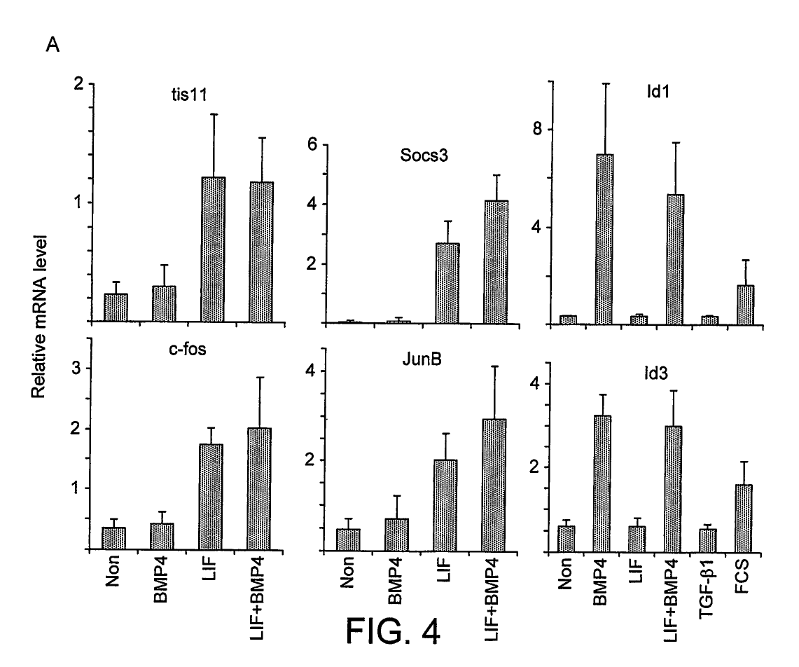

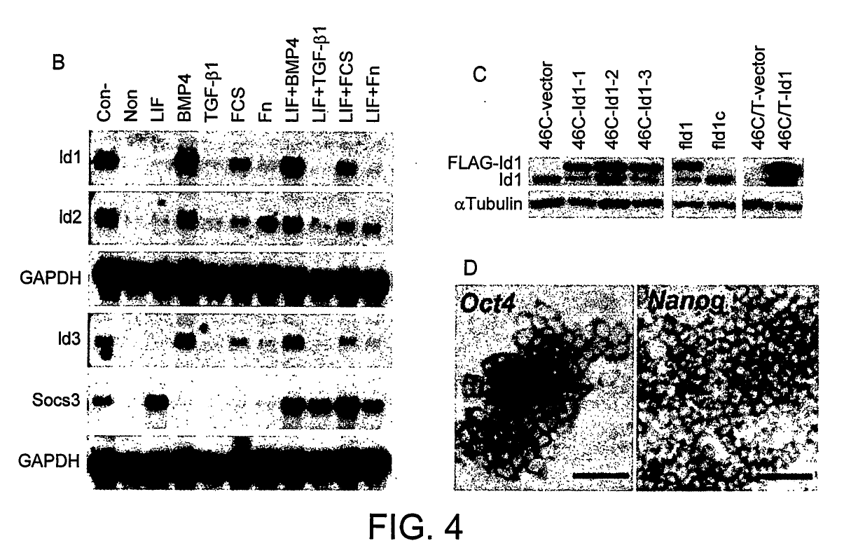

FIG. 4 shows expression and function of Ids in ES cells;

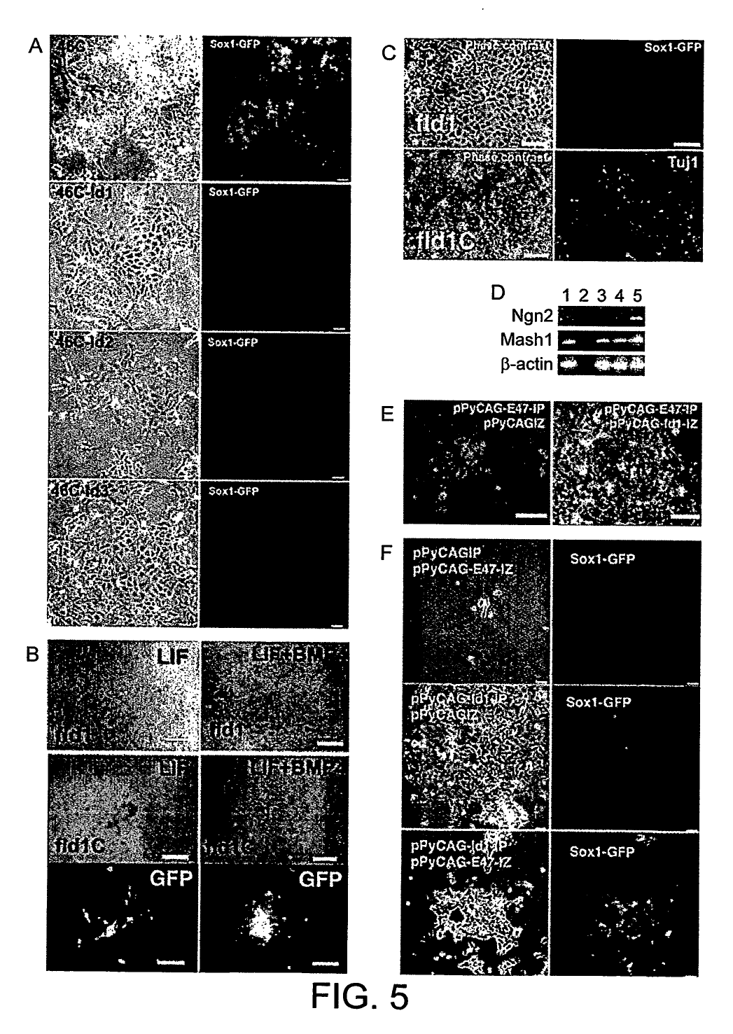

FIG. 5 shows Id suppresses neural differentiation and is required for ES cell self-renewal;

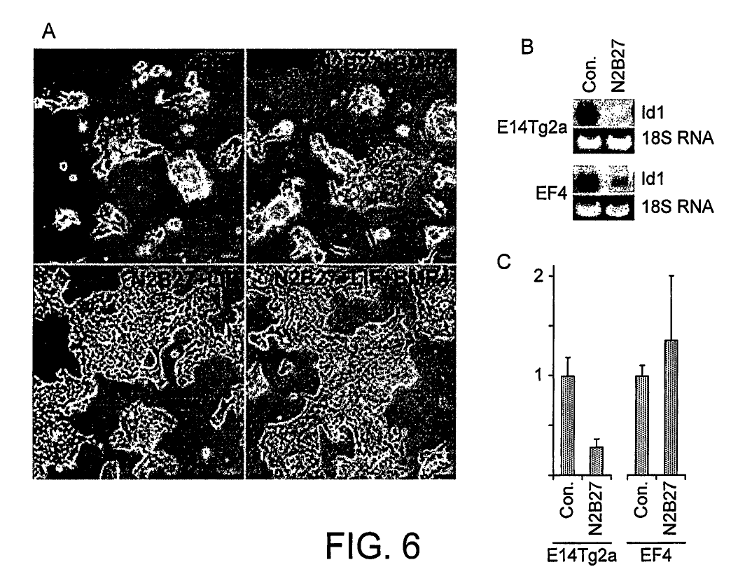

FIG. 6 shows Nanog bypasses requirement for BMP/serum to induce Id; and

FIG. 7 shows cooperative lineage restriction by BMP/Id and LIF/STAT3

In more detail, and referring to the examples set out below, FIG. 1 shows LIF plus BMP sustain ES cell self-renewal in serum-free medium:—

A. Phase contrast and fluorescent images of Oct4-GiP cells cultured in N2B27 with the indicated factors. TuJ1 immunostaining detects neuronal differentiation, green fluorescence reflects activity of the Oct4 promoter in undifferentiated ES cells. Bar: 50 μm

B. Plot of cumulative Oct4-GFP positive undifferentiated ES cell numbers during progressive passaging in conventional medium with FCS plus LIF or in N2B27 with LIF (10 ng/ml) plus BMP4 (10/ng/ml). Cultures were passaged every 48 hours using cell dissociation buffer and replated at 4×10⁵ cells per 10 cm² well. The number of GFP positive cells was determined by FACS analysis at each passage.

C. RT-PCR analysis of Oct4, Nanog, T (brachyury), and Sox1 mRNAs in (1) ES cells in N2B27 with LIF plus BMP for 6 passages, (2) ES cells cultured in serum with LIF, (3) day 8 embryoid bodies (4) day 8 embryoid bodies with retinoic acid treatment.

FIG. 2 shows clonogenicity, potency and derivation of ES cells in N2B27 with LIF plus BMP:—

A. CAG-taugfp transfectant colony isolated by electroporation of E14Tg2a cells and selection in puromycin.

B. Single CAG-taugfp transfectant ES cell and derivative colony.

C. Mid-gestation foetal chimaera produced from TP6.3 ES cells after 6 passages in N2B27 with LIF plus BMP. GFP fluorescence marks ES cell progeny.

D. Male chimaera from CAG-taugfp transfected ES cell with C57Bl/6 mate and offspring. Agouti coat colour denotes ES cell origin of offspring.

E. Colony of first passage SF1 ES cells derived in N2B27 with LIF plus BMP.
Chimaeras were generated from SF1 ES cells
   Bar: 50 μm

FIG. 3 shows BMP signalling in ES cells:—

A. Reverse transcription-PCR analysis of RNA samples from Oct-GiP cells (1) in N2B27 with LIF plus BMP, passage 6, (2) in serum plus LIF, no reverse transcriptase control (3) in serum plus LIF, (4) day 1 after plating in N2B27 without LIF or BMP, (5) day 5 without LIF or BMP.

B. Immunoblots showing Smad1, erk and p38 response to mock treatment (non) or stimulation with LIF, BMP, or LIF plus BMP for 15 minutes or 1 hour after overnight culture in N2B27.

C. Immunoblot showing STAT3 tyrosine phosphorylation response to LIF, BMP, and LIF plus BMP.

D. Smad7 episomal transfectants differentiate and express neural precursor (Sox1-GFP) and neuronal (TuJ) markers in the presence of serum and LIF

E. SB203580 (30 μM) p38 inhibitor does not suppress either self-renewal in LIF plus BMP or neural differentiation in LIF alone. Oct4-GFP marks undifferentiated ES cells and TuJ1 immunostaining identifies neurons.

F. Co-immunoprecipitation of active Smad1 and STAT3 in ES cells. Left panel: FLAG immunoprecipitates following transfection with FLAG-tagged Smad1. Right panel: STAT3 immunoprecipitates from non-manipulated ES cells. Cells were stimulated as indicated for 1 hour.

Bar: 50 μm

FIG. 4 shows expression and function of Ids in ES cells:

A. LightCycler reverse transcription PCR analyses of gene induction in response to LIF, BMP, or LIF+BMP. ES cells were cultured overnight in N2B27 alone, then stimulated for 45 minutes.

B. Northern hybridisation of Id mRNA expression in Oct4-GiP cells. Con: steady state ES cells maintained in serum containing medium plus LIF. Lanes 2-11 cells cultured overnight in N2B27 without factors then stimulated as indicated for 45 minutes. Fn, fibronectin.

C. Steady state level of Id1 protein in 46C ES cells transfected with vector alone and cultured in serum-containing medium with LIF, and overexpression in Id1 and fld1 stable integrant clones and after episomal supertransfection of 46C/T cells. The latter blot was exposed for only 10 seconds. Transfected Id1 is FLAG tagged and therefore has retarded migration compared with endogenous Id1.

D. In situ hybridisation of Nanog and Oct4 mRNA in Id1 stable integrant ES cell colonies cultured in N2B27 plus LIF. Equivalent results were obtained with Id2 and Id3 transfectants. Bar: 50 μm

FIG. 5 shows Id suppresses neural differentiation and is required for ES cell self-renewal:—

A. Phase contrast and GFP fluorescence images of vector and Id3 stable integrant 46C clones after 6 days differentiation in N2B27 without added factors. Id1 and Id2 transfectants showed similar suppression of neural differentiation.

B. Upper panels: fld1 transfectant 46C cells form self-renewing colonies in N2B27 with LIF alone. Middle panels: after Cre excision fld1C cells differentiate in LIF and require LIF plus BMP for ES colony formation. Lower panels: GFP expression in fld1C colonies driven by the constitutive CAG unit after excision of the floxed Id1-STOP cassette.

C. fld1 cells undergo non-neural differentiation on withdrawal of LIF in N2B27 and do not activate Sox1-GFP or express TuJ. After Cre excision, fld1C cells show restored differentiation of TuJ positive neuronal cells. (Sox1-GFP cannot be specifically detected in fldC cells due to the constitutive activation of GFP)

D. Reverse transcription PCR analysis of mash1 and ngn2 expression in ES cells and during neural differentiation. Samples as in FIG. 3A.

E. Overexpression of E47 blocks ES cell self-renewal, which can be rescued by increased Id1. 46C/T ES cells were supertransfected with E47 or co-supertransfected with E47 plus Id1 episomal expression vectors and cultured for 6 days under dual puromycin and zeocin selection in serum-containing medium with LIF.

F. Increased E47 overcomes Id1 suppression of neural differentiation. 46C/T ES cells were supertransfected as in E then 24 hours after transfection transferred into N2B27 without added factors and cultured for 6 days under dual selection.

Bar: 50 μm

FIG. 6 shows Nanog bypasses requirement for BMP/serum to induce Id:—

A. EF4C cells were cultured for 6 days in N2B27 or in N2B27 plus BMP. EF4 Nanog transfectants were cultured under the indicated conditions for 6 passages and then photographed. Bar: 50 μm

B and C. Northern hybridisation of Id1 and Id3 mRNAs in E14Tg2a parental ES cells and EF4 Nanog transfectants in serum plus LIF (Con) or overnight in N2B27 without factors, and mRNA levels.

FIG. 7 schematically shows cooperative lineage restriction by BMP/Id and LIF/STAT3:—

ES cell self-renewal requires suppression of lineage commitment. Id genes induced by BMP or other signals blockade entry into neural lineages, which is otherwise only partially prevented by LIF/STAT3. In parallel, the capacity of BMP to induce mesodermal and endodermal differentiation is constrained by STAT3, probably involving direct as well as indirect mechanisms. Withdrawal of LIF therefore results in a switch in BMP action from supporting self-renewal to promoting lineage commitment.

In the sequence listing for this invention the following SEQ ID No:s correspond to the following:—

1 amino acid sequence for mouse Id3

2 amino acid sequence for rat Id3

3 amino acid sequence for canine Id3

4 amino acid sequence for human Id3

5 protein transduction domain from Tat

6 protein transduction domain from antennapedia

7 Tat-human Id 3 fusion

8 antennapedia-human Id 3 fusion

9 mouse Id 3-antennapedia fusion

### EXAMPLES

Foetal calf serum is important for viability of undifferentiated ES cells in minimal media (Wiles and Johansson, 1999). However, in enriched basal media containing N2 and B27 supplements ES cell viability remains high (Ying and Smith, 2003). This allowed us to examined whether LIF is capable of driving continuous cycles of self-renewal in the absence of serum factors.

In N2B27 medium alone adherent ES cells efficiently convert into Sox1 positive neural precursors (Ying et al., 2003). LIF reduces but does not eliminate neural differentiation under these conditions. Upon successive passaging in N2B27 medium plus LIF we found that following an initial increase, the number of undifferentiated ES cells reached a plateau and then began to decline after 2-3 passages. This finding was reproduced with several different ES cell lines. Many cells in these cultures had morphology of neural precursors or immature neurons. Neural differentiation was confirmed by activation of the Sox1-GFP neural reporter in 46C ES cells (Ying et al., 2003). These observations indicate that additional signalling pathways to LIF/STAT3 are required to promote ES cell self-renewal and in particular to suppress neural determination.

BMPs are well known anti-neural factors in vertebrate embryos (Wilson and Hemmati-Brivanlou, 1995; Wilson and Edlund, 2001) and have been shown to antagonise neural differentiation of ES cells (Tropepe et al., 2001; Ying et al., 2003). BMP alone promotes differentiation of ES cells into non-neural fates (Johansson and Wiles, 1995; Wiles and Johansson, 1999; Ying et al., 2003) and therefore initially appears unlikely as a candidate self-renewal factor. However, we examined whether addition of BMP might contribute to an inhibition of differentiation in conjunction with co-stimulation by LIF. We found that the combination of LIF plus BMP4 (or BMP2) enhanced self-renewal resulting in highly pure populations of undifferentiated ES cells after 2 or 3 passages in N2B27 (FIG. 1A). These cultures could subsequently be expanded for multiple passages with no deterioration in growth rate or viability and no neural differentiation (FIG. 1A, B). This response was observed in each of 11 different ES cell lines, originating from three independent derivations. The representation of Oct4 positive undifferentiated cells and the population doubling time were slightly higher than obtained in serum plus LIF (FIG. 1B). ES cell status was confirmed by expression of SSEA-1 and alkaline phosphatase (not shown), and of mRNAs for ES cell specific transcription factors Nanog and Oct4 with absence of markers of mesoderm (T) and neuroectoderm (Sox1) (FIG. 1C).

The N2 and B27 components improve viability but are not essential for self-renewal. In basal medium supplemented only with transferrin, self-renewal and undifferentiated ES cell expansion can be sustained for multiple passages by LIF plus BMP, but not by LIF alone. The requirement for BMP is therefore not induced by a component in B27.

We tested the BMP relative growth and differentiation factor-6 (GDF-6) and found that it similarly supported ES cell self-renewal in the presence of LIF (FIG. 1A). This is not a general feature of the TGF-β superfamily, however, but is restricted to BMP receptor ligands. TGF-β1 had no discernible effect on ES cells, whilst activin increased viability and/or proliferation but did not suppress differentiation.

To test the efficiency of ES cell propagation supported by LIF plus BMP, we undertook electroporation and selection of stable transfectants. Colonies stably expressing tauGFP were readily isolated (FIG. 2A) and could be amplified into bulk cultures demonstrating the feasibility of using this serum-free system in genetic manipulation protocols.

Self-renewal of isolated ES cells was then investigated. Single ES cells were transferred to 96-well plates in N2B27 with addition of LIF only or of LIF plus BMP4 (FIG. 2B). A single colony that formed in the presence of LIF alone contained a high proportion of differentiated cells and could not be expanded further. In contrast, undifferentiated colonies formed in 12/192 wells in LIF plus BMP4 and 10 of these were amplified without serum (Table).

ES cells cultured in LIF plus BMP maintained a diploid chromosome complement after multiple generations. They also retained differentiation potential. Withdrawal of both LIF and BMP resulted in neural differentiation. Removal of LIF with retention of BMP caused differentiation into sheets of flattened epithelial-like cells. Thus the self-renewal response to BMP remains dependent on continuous LIF signalling.

The definitive functional attribute of mouse ES cells is their capacity to re-enter embryonic development and contribute to the full repertoire of differentiated tissues in chimaeric mice. We injected GFP reporter ES cells into mouse blastocysts after propagation in N2B27 with LIF plus BMP for 3 weeks. Analysis at mid-gestation identified several chimaeras with high ES cell contributions to a range of tissues (FIG. 2C). As a more rigorous test we used ES cells transfected with taugfp and selected and expanded in LIF plus BMP. Liveborn chimaeras were obtained and two male animals transmitted the ES cell genome (FIG. 2D).

Derivation of ES Cells without Feeders or Serum.

We investigated whether the response to BMP may be an adaptation of established ES cells to culture or is manifest during the initial stages of ES cell derivation. We plated blastocysts in N2B27 supplemented with BMP plus LIF. After several days expanded inner cell masses (ICMs) were dissociated and replated in the same culture conditions. In initial trials ES cell colonies were not obtained following ICM dissociation after 5-6 days in culture, the standard timing for ES cell derivation (Nichols et al., 1990; Robertson, 1987). However, in the absence of serum and presence of BMP the ICM exhibits reduced growth and more rapid onset of overt differentiation. Therefore we subsequently dissociated the ICM after only 4 days of blastocyst culture in LIF only and added BMP4 on replating. Under these conditions primary ES cell colonies did form (FIG. 2E). These could be passaged and expanded as morphologically undifferentiated ES cells. One line (SF1) was characterised further. Upon withdrawal of LIF and BMP, SF1 ES cells underwent neural differentiation in vitro. Moreover, SF1 cells produced extensively chimaeric mice (FIG. 2F). Twelve chimaeras were all male, indicative of sex conversion by highly contributing XY ES cells (Bradley et al., 1984).

Hence, we derived ES cells in accordance with the invention by culturing replated cells in the presence of gp130 signalling and an activator of downstream signalling from a receptor of the TGF-β superfamily.

Undifferentiated ES Cells Express Functional BMP Signalling Machinery.

Single cell cloning and the near-complete absence of differentiation in LIF plus BMP cultures suggested to us that the effect of BMP is likely to be directly on ES cells rather than mediated via differentiated progeny. However, previous studies reporting BMP receptor expression and BMP responsiveness during ES cell differentiation (Adelman et al., 2002; Hollnagel et al., 1999) have not established whether ES cells in the undifferentiated state can actually respond to BMP. To confirm this we used selection for activity of an Oct4 transgene (Ying et al., 2002) to purify undifferentiated cells for RNA and protein analyses.

BMPs act through heterodimers of type 1 and type II serine/threonine kinase receptors (Shi and Massague, 2003). Undifferentiated ES cells show little or no type I BmprIb mRNA, but express both type I BmprIa and type I BmprII receptor mRNAs (FIG. 3A). BMP4 and GDF6 transcripts are also readily detectable in undifferentiated ES cells. The principal effectors downstream of the BMP receptors are the Smad transcription factors (Attisano and Wrana, 2002; von Bubnoff and Cho, 2001). R-Smads 1, 5 and 8 are recruited to and phosphorylated by the active BMP receptor complex and then combine with Smad4 and translocate to the nucleus. We investigated Smad activation by immunoblotting using antibody specific for the active serine phosphorylated form of Smad1. Increased phosphorylation of Smad1 in undifferentiated ES cells is apparent after BMP4 addition (FIG. 3B). BMP stimulation also enhances the basal activation of p38 and, by one hour, of erk mitogen-activated protein kinases (FIG. 3B). These data establish that undifferentiated ES cells possess the signal transduction machinery for responsiveness to BMP stimulation and furthermore that they may have the potential for autocrine stimulation via BMP4 and GDF production.

BMP Supports Self-Renewal through Smad Activation.

The self-renewal action of LIF is mediated via the transcription factor STAT3 (Matsuda et al., 1999; Niwa et al., 1998). BMP alone does not activate STAT3 measured by phosphorylation of tyrosine 705 (FIG. 3C). Nor does it increase STAT3 activation by LIF. Erk activation downstream of gp130 is not required for ES cell self-renewal but appears to be a pro-differentiative signal (Burdon et al., 1999a). Thus reduced erk activity facilitates ES cell derivation (Buehr and Smith, 2003) and promotes self-renewal (Burdon et al., 1999b). Erk activation in response to LIF was not appreciably inhibited by the presence of BMP, however (FIG. 3B). These data indicate that BMP does not modulate gp130 signal transduction in ES cells, implying that a BMP signalling pathway contributes directly to self-renewal.

We introduced the inhibitory Smad family members, Smad6 and Smad7 (Shi and Massague, 2003; von Bubnoff and Cho, 2001), into ES cells to antagonise BMP signalling. Cells were transfected and grown up under puromycin selection in the presence of serum and LIF. Smad6 or Smad7 expression vectors yielded fewer and smaller ES cell colonies relative to transfections with empty vector. Furthermore Smad6 and even more so Smad7 transfectants expanded poorly after passaging. A high level of differentiation was evident in the transfected cell populations. Neural differentiation is normally suppressed by serum in adherent cultures, but was readily apparent after Smad7 transfection (FIG. 3D).

In addition to blocking Smad activity, Smad6/7 can also inhibit the TAK/p38 pathway downstream of BMPR (Kimura et al., 2000). To assess the potential contribution of p38 in ES cells we used the specific inhibitor SB203580 (Cuenda et al., 1995). This reagent had no noticeable effect on the capacity of BMP to support self-renewal (FIG. 3E). In LIF only, SB203580 did not alter the balance between self-renewal and neural differentiation, but appeared to enhance overall cell viability, suggesting that in ES cells as in other cell types p38 is pro-apoptotic (Kimura et al., 2000). The Smad pathway is therefore the likely transducer of the self-renewal signal.

A mechanism of cooperative transcriptional regulation between Smad and STAT3 has been characterised in neuroepithelial cells (Nakashima et al., 1999; Sun et al., 2001). This involves formation of a ternary complex bridged by the ubiquitous transcriptional co-activator p300 and results in synergistic activation of glial-specific promoters. We investigated whether a complex containing STAT3 and Smads may be formed in ES cells stimulated with LIF plus BMP. Immunoprecipation following transfection with FLAG-tagged Smad1 indicated that activated STAT3 and Smad1 may co-localise (FIG. 3F). This conclusion was corroborated by co-immunoprecipitation of endogenous phosphorylated Smad1 and STAT3 following LIF plus BMP stimulation (FIG. 3F).

BMP Target Genes in ES Cells.

To effect ES cell self-renewal, BMP/Smad and LIF/STAT3 signalling could operate in parallel on distinct target genes and/or may converge on common target genes, for example via the ternary complex with p300. We used real time RT-PCR to survey candidate genes for induction by LIF, BMP, or LIF plus BMP in Oct-selected ES cells (FIG. 4A). Two known LIF targets tis11 and c-fos showed no response to BMP. Two others, junB and in particular socs3, appeared to be more highly induced by LIF in the presence of BMP. These data suggest that a subset of STAT3 target genes may be responsive to co-stimulation with BMP. However, neither JunB nor Socs3 are candidates for effectors of self-renewal: junB null ES cells show no defects (Schorpp-Kistner et al., 1999), and SOCS3 functions as a negative feedback regulator of gp130 signalling (Schmitz et al., 2000) that blocks self-renewal when overexpressed.

We also examined expression of Id genes, which encode negative bHLH factors and have been shown to be induced by BMP/Smad in neuroepithelial cells (Nakashima et al., 2001) and C2C12 myoblasts (Lopez-Rovira et al., 2002). Id mRNA induction by BMP has also been reported in differentiating ES cell cultures (Hollnagel et al., 1999). We found that Id1 and Id3 are strongly induced by BMP (and GDF, data not shown), but not by LIF (FIG. 4A). Northern hybridisation confirmed these findings and extended them to Id2 (FIG. 4B). Neither activin (data not shown) nor TGF-β1 induce Id gene expression indicating that this response is specific to Smads downstream of the BMP receptor.

The Id genes are also induced by foetal calf serum and by fibronectin, although to a lesser extent than by BMP (FIG. 4B). ES cells cultured in serum show readily detectable steady state amounts of Id mRNAs. We examined whether fibronectin, which induces Id2 and Id3, could replace BMP in N2B27 cultures. Soluble fibronectin in combination with LIF could expand undifferentiated Oct4-GiP cells for at least 10 passages, although with more differentiation and slower population expansion than in BMP.

Constitutive Id Bypasses BMP or Serum Requirements for ES Cell Self-Renewal.

We hypothesized that Id induction may provide a specific restriction of neural differentiation to complement the self-renewal activity of STAT3. Accordingly we prepared expression constructs for Id1, Id2 and Id3 and introduced these into ES cells. Colonies were readily recovered by both episomal supertransfection and conventional stable integration. For Id1, elevated protein expression was confirmed by immunoblotting (FIG. 4C). Overexpression of the transgene appears to be associated with a reduction in endogenous Id1 protein, implying operation of a feedback or autoregulatory loop.

Forced Id expression did not impair ES cell self-renewal nor block differentiation in the presence of serum. Under these conditions the transfectants were not overtly different from parental ES cells or empty vector transfectants. In contrast, in serum-free N2B27, Id transfectants whilst remaining LIF-dependent, were liberated from requirement for BMP. These cells proliferated in LIF alone as rapidly and with as little differentiation as parental ES cells in LIF plus BMP. The cultures could be passaged multiple times with no change in undifferentiated morphology or factor dependence. The ES cell phenotype was confirmed by expression of Oct4 and Nanog mRNAs (FIG. 4D). As a rigorous test of the capacity of Id expression to substitute for serum or BMP/GDF, we plated single cells in N2B27. Undifferentiated passageable colonies formed in LIF alone with comparable frequency (10%) to colony formation from isolated cells in LIF plus BMP (Table).

Id Proteins Exert a Lineage-Specific Block on ES Cell Differentiation.

In our cultures, LIF is essential for self-renewal of Id transfectants because Ids do not impose a complete block on ES cell differentiation. If LIF is withdrawn in serum-containing medium, Id transfectant cells differentiate as parental ES cells. In adherent culture they produced mostly flattened epithelial-like cells with some fibroblasts. On aggregation they formed embryoid bodies with activation of mesodermal (T) and endodermal (Hnf4) marker expression (data not shown) and developed spontaneous contractility indicative of cardiomyocyte differentiation. However, in N2B27 in the absence of LIF, Id transfectants behaved differently from other ES cells. Neural differentiation, assessed by morphology and by activation of Sox1-GFP was minimal (FIG. 5A). Instead the transfectants differentiated into sheets of flattened epithelioid cells, similar to parental ES cells exposed to BMP alone (cf FIG. 1A).

We prepared a revertable expression construct to test whether self-renewal and blockade of neural differentiation are dependent on continuous Id expression. We generated 46C ES cells expressing floxed Id1 (fld1 cells) and subsequently a Cre treated derivative clone (fld1C) in which the Id1 transgene had been excised. After Cre excision, fld1C cells show absence of FLAG-Id1 and restored levels of endogenous Id1 (FIG. 4C). fld1 and fld1C cells were plated at clonal density in N2B27 with LIF or LIF plus BMP. fld1 cells formed stem cell colonies efficiently in LIF alone but this ability was lost in fld1C cells which produced only differentiated cells in LIF without BMP (FIG. 5B). In N2B27 alone, fld1 cells underwent non-neural differentiation whereas fld1C cells behaved in identical fashion to parental ES cells, generating a high proportion of TuJ positive neurons (FIG. 5C).

These observations indicate that Id expression specifically blocks neural lineage commitment and diverts differentiating ES cells into alternative fates, much as observed for BMP treatment in the absence of LIF (Ying et al., 2003). Id expressing ES cells are thus wholly dependent on LIF/STAT3 for inhibition of non-neural lineage commitment and maintenance of pluripotency.

The neurogenic bHLH transcription factors are known to be antagonised by Id proteins in the developing CNS (Lyden et al., 1999). In vivo these bHLH factors have not been reported prior to neurulation. However, cultured ES cells show expression of mRNAs expected to be found only in differentiating lineages (Ramalho-Santos et al., 2002). We therefore investigated the potential expression of two bHLH genes, mash1 and neurogenin2, in Oct4 selected ES cells. Whilst neurogenin2 mRNA is not detectable above background levels, mash1 mRNA appears relatively abundant (FIG. 5D). We propose therefore that Id expression may be necessary to prevent continuous neural differentiation of ES cells triggered by precocious expression of mash1 and other pro-neural bHLH factors. Such action may also encompass non-bHLH partners such as Pax and Ets factors (Norton, 2000).

Id proteins bind to ubiquitous HLH factors, the E proteins, with high avidity (Norton, 2000). Overexpression of either will sequester and block activity of the other. To assess whether Id proteins may normally be required for ES cell propagation we overexpressed the E47 protein by episomal supertransfection either alone or in co-transfection with Id1 or Id3. E47 singly or in co-transfection with empty vector yielded few, very small and sickly colonies (FIG. 5E). In contrast, healthy ES cell colonies were generated from co-transfection of E47 and Id vectors. Co-transfectant colonies appeared indistinguishable in serum-containing medium from cells transfected with Id alone or with empty vector. This suggests that increased E47 is not intrinsically toxic but has a specific growth inhibitory action due to sequestration of Id. A certain level of free Id may by required for ES cell propagation as observed in other cell types (Norton, 2000). When transferred to N2B27 without LIF or BMP, the co-transfectants underwent neural rather than non-neural differentiation, shown by activation of Sox1-GFP (FIG. 5F). Thus E47 neutralises the neural suppression effect of Id. This is consistent with the suggestion that Id acts to limit availability of E proteins for partnering with proneural bHLH factors.

Nanog can Bypass Requirements for BMP or Serum.

Increased levels of the variant homeodomain protein Nanog render ES self-renewal independent of LIF/STAT3 in the presence of serum (Chambers et al., 2003). We examined whether LIF and/or BMP are required for self-renewal of Nanog overexpressing ES cells in N2B27. FIG. 6A shows that EF4 cells expressing a floxed Nanog transgene can be propagated in N2B27 without either LIF or BMP. This behaviour is directly attributable to Nanog, since derivative EF4C cells in which the Nanog transgene has been excised by Cre recombinase rapidly undergo neural differentiation. Addition of BMP alone has no apparent effect on EF4 cells, unless cultures are maintained without passage for more than 6 days when some differentiation becomes apparent (see Discussion). On addition of LIF, with or without BMP, EF4 cells adhere more evenly to the culture dish (FIG. 6A) and the population doubling rate increases. This accords with previous indications of combinatorial effects of LIF/STAT3 and Nanog in ES cells (Chambers et al., 2003).

Since Nanog renders BMP or serum stimulation redundant, we asked whether EF4 cells express Ids. After overnight culture in N2B27 without LIF or BMP, expression of Id1 and Id3 was markedly down-regulated in parental E14Tg2a cells. By contrast, in EF4 cells Id1 mRNA was reduced though still appreciable, and Id3 mRNA actually increased (FIG. 6B). Thus overexpression of Nanog can be used to maintain a substantial level of Id expression constitutively.

Experimental Procedures

ES Cell Culture

ES cells were maintained without feeder cells. For serum-free culture, ES cells were plated onto gelatin-coated plates in N2B27 medium (Ying and Smith, 2003) supplemented with 10 ng/ml LIF (Sigma) and 10 ng/ml BMP4 or 200 ng/ml GDF6 (R&D Systems). Cells were passaged every 2-4 days using either enzyme-free Cell Dissociation Buffer (Invitrogen) or 0.025% trypsin/1% chicken serum. Dissociated cells were harvested in N2B27 and pelleted. Supernatant was aspirated and the cell pellet resuspended in N2B27 and replated directly. For single cell cloning, a finely drawn Pasteur pipette pre-loaded with N2B27 was used to pick individual cells into 10 μl drops. Drops were then singly transferred to 96-well plates pre-loaded with 150 μl N2B27 per well with LIF or LIF plus BMP4. After 8 days, ES cell colonies were identified and passaged. To produce chimaeras, ES cells were injected into C57Bl/6 blastocysts. Germline transmission was tested by mating male chimaeras with C57Bl/6 females.

Derivation of ES Cells in Serum Free Medium

Strain 129 mice were ovariectomised on the third day of pregnancy and embryos in diapause flushed 4 days later (Nichols et al., 1990). Intact blastocysts were plated on gelatin-coated plastic in N2B27 supplemented with LIF (10 ng/ml). After 3-6 days the central mass of each explant was picked, rinsed in PBS and placed in a drop of trypsin for a few minutes. The cell mass was picked up in a finely drawn out Pasteur pipette pre-loaded with medium, ensuring minimal carry over of trypsin, and expelled with gentle trituration into a fresh well in N2B27 supplemented with LIF and BMP4 (10 ng/ml). Resultant primary ES cell colonies were individually passaged into wells of a 96 well plate. Thereafter, cells were expanded by trypsinisation of the entire culture with centrifugation and aspiration before replating.

RNA Analyses

Oct4GiP ES cells (Ying et al., 2002) were cultured in the presence of puromycin for 4-6 days to eliminate differentiated cells. Purified ES cells were cultured in complete medium plus LIF for 24 hours then washed once with PBS and transferred to N2B27 medium overnight prior to stimulation for 45 min. with 20 ng/ml LIF, 50 ng/ml BMP4, LIF plus BMP4, 10 ng/ml TGF-β1 (all R&D Systems) or 15% FCS. Quantitative RT-PCR was carried out using the LightCycler Instrument (Roche). Data were normalised relative to Oct4 amplification. Primer pairs and reaction conditions are available upon request. Northern hybridizations were carried out on 5 μg aliquots of total RNA.

Plasmid Construction and Transfection

Smad6 and Smad7 plasmids were kindly provided by Hitoshi Niwa and FLAG-tagged Id1 by Tetsuya Taga. Mouse Id2, Id3 and E47 open reading frames (ORFs) were amplified by PCR, cloned into pCR2.1, and verified mutation-free by sequence analysis. Expression vectors were introduced into ES cells episomally or by stable integration. Floxed Id1 and Cre-excised derivative ES cell lines were derived using the strategy described by Chambers et al., 2003.

Immunochemistry

Pre-selected Oct4GiP ES cells were transferred to N2B27 medium overnight prior to stimulation with LIF (20 ng/ml), BMP4 (50 ng/ml) or LIF plus BMP4 for 15 min or 1 hour. Phosphorylated stat3, smad1, erk1/2 and p38 were detected by immunoblotting (Cell Signaling Technology). Cell lysis and immunoprecipitation (Nakashima et al., 1997) employed anti-FLAG (Sigma) or anti-Stat3 (Transduction Labs). Immunostaining was performed as described (Ying et al., 2003)

### REFERENCES

Adelman, C. A., Chattopadhyay, S., and Bieker, J. J. (2002). The BMP/BMPR/Smad pathway directs expression of the erythroid-specific EKLF and GATA1 transcription factors during embryoid body differentiation in serum-free media. Development 129, 539-549.
Attisano, L., and Wrana, J. L. (2002). Signal transduction by the TGF-beta superfamily. Science 296, 1646-1647.
Baonza, A., de Celis, J. F., and Garcia-Bellido, A. (2000). Relationships between extramacrochaetae and Notch signalling in Drosophila wing development. Development 127, 2383-2393.
Beddington, R. S. P., and Robertson, E. J. (1989). An assessment of the developmental potential of embryonic stem cells in the midgestation mouse embryo. Development 105, 733-737.
Benezra, R. (2001). Role of Id proteins in embryonic and tumor angiogenesis. Trends Cardiovasc Med 11, 237-241.
Bradley, A., Evans, M. J., Kaufman, M. H., and Robertson, E. (1984). Formation of germ-line chimaeras from embryo-derived teratocarcinoma cell lines. Nature 309, 255-256.
Brook, F. A., and Gardner, R. L. (1997). The origin and efficient derivation of embryonic stem cells in the mouse. Proc. Natl. Acad. Sci. USA 94, 5709-5712.
Buehr, M., and Smith, A. (2003). Genesis of embryonic stem cells. Phil. Trans. R. Soc., B in press.
Burdon, T., Chambers, I., Niwa, H., Stracey, C., and Smith, A. G. (1999a). Signalling mechanisms regulating self-renewal and differentiation of pluripotent embryonic stem cells. Cells Tissues Organs 165, 131-143.
Burdon, T., Stracey, C., Chambers, I., Nichols, J., and Smith, A. (1999b). Suppression of SHP-2 and ERK signalling promotes self-renewal of mouse embryonic stem cells. Dev. Biol. 210, 30-43.
Chambers, I., Colby, D., Robertson, M., Nichols, J., Lee, S., Tweedie, S., and Smith, A. (2003). Functional expression cloning of Nanog, a pluripotency sustaining factor in embryonic stem cells. Cell 113, 643-655.
Cuenda, A., Rouse, J., Doza, Y. N., Meier, R., Cohen, P., Gallagher, T. F., Young, P. R., and Lee, J. C. (1995). SB 203580 is a specific inhibitor of a MAP kinase homologue which is stimulated by cellular stresses and interleukin-1. FEBS Lett 364, 229-233.
Harland, R. (2000). Neural induction. Curr Opin Genet Dev 10, 357-362. Hollnagel, A., Oehimann, V., Heymer, J., Ruther, U., and Nordheim, A. (1999). Id genes are direct targets of bone morphogenetic protein induction in embryonic stem cells. J. Biol. Chem. 274, 19838-19845.
Hu, M., Krause, D., Greaves, M., Sharkis, S., Dexter, M., Heyworth, C., and Enver, T. (1997). Multilineage gene expression precedes commitment in the hemopoietic system. Genes Dev. 11, 774-785.
Jen, Y., Weintraub, H., and Benezra, R. (1992). Overexpression of Id protein inhibits the muscle differentiation program: in vivo association of Id with E2A proteins. Genes Dev. 6, 1466-1479.
Johansson, B. M., and Wiles, M. V. (1995). Evidence for involvement of activin A and bone morphogenetic protein 4 in mammalian mesoderm and hematopoietic development. Mol. Cell. Biol. 15, 141-151.
Kawasaki, H., Mizuseki, K., Nishikawa, S., Kaneko, S., Kuwana, Y., Nakanishi, S., Nishikawa, S. I., and Sasai, Y. (2000). Induction of midbrain dopaminergic neurons from ES cells by stromal cell-derived inducing activity. Neuron 28, 31-40.
Kiger, A. A., Jones, D. L., Schulz, C., Rogers, M. B., and Fuller, M. T. (2001). Stem cell self-renewal specified by JAK-STAT activation in response to a support cell cue. Science 294, 2542-2545.
Kimura, N., Matsuo, R., Shibuya, H., Nakashima, K., and Taga, T. (2000). BMP2-induced apoptosis is mediated by activation of the TAK1-p38 kinase pathway that is negatively regulated by Smad6. J. Biol. Chem. 275, 17647-17652.
Kretzschmar, M., Doody, J., and Massague, J. (1997). Opposing BMP and EGF signalling pathways converge on the TGF-beta family mediator Smad1. Nature 389, 618-622.
Lopez-Rovira, T., Chalaux, E., Massague, J., Rosa, J. L., and Ventura, F. (2002). Direct binding of Smad1 and Smad4 to two distinct motifs mediates bone morphogenetic protein-specific transcriptional activation of Id1 gene. J. Biol. Chem. 277, 3176-3185.
Lyden, D., Young, A. Z., Zagzag, D., Yan, W., Gerald, W., O'Reilly, R., Bader, B. L., Hynes, R. O., Zhuang, Y., Manova, K., and Benezra, R. (1999). Id1 and Id3 are required for neurogenesis, angiogenesis and vascularization of tumour xenografts. Nature 401, 670-677.
Matsuda, T., Nakamura, T., Nakao, K., Arai, T., Katsuki, M., Heike, T., and Yokota, T. (1999). STAT3 activation is sufficient to maintain an undifferentiated state of mouse embryonic stem cells. Embo J 18, 4261-4269.
Mishina, Y., Suzuki, A., Ueno, N., and Behringer, R. R. (1995). Bmpr encodes a type I bone morphogenetic protein receptor that is essential for gastrulation during mouse embryogenesis. Genes Dev. 9, 3027-3037.
Nakashima, K., Narazaki, M., and Taga, T. (1997). Overlapping and distinct signals through leptin receptor (OB-R) and a closely related cytokine signal transducer, gp130. FEBS Lett 401, 49-52.
Nakashima, K., Takizawa, T., Ochiai, W., Yanagisawa, M., Hisatsune, T., Nakafuku, M., Miyazono, K., Kishimoto, T., Kageyama, R., and Taga, T. (2001). BMP2-mediated alteration in the developmental pathway of fetal mouse brain cells from neurogenesis to astrocytogenesis. Proc. Natl. Acad. Sci. USA 98, 5868-5873.
Nakashima, K., Yanagisawa, M., Arakawa, H., Kimura, N., Hisatsune, T., Kawabata, M., Miyazono, K., and Taga, T. (1999). Synergistic signaling in fetal brain by STAT3-Smad1 complex bridged by p300. Science 284, 479-482.
Nichols, J., Chambers, I., Taga, T., and Smith, A. G. (2001). Physiological rationale for responsiveness of mouse epiblast and embryonic stem cells to gp130 cytokines. Development 128, 2333-2339.
Nichols, J., Evans, E. P., and Smith, A. G. (1990). Establishment of germ-line competent embryonic stem (ES) cells using differentiation inhibiting activity. Development 110, 1341-1348.
Nishinakamura, R., Matsumoto, Y., Matsuda, T., Ariizumi, T., Heike, T., Asashima, M., and Yokota, T. (1999). Activation of Stat3 by cytokine receptor gp130 ventralizes Xenopus embryos independent of BMP-4. Dev. Biol. 216, 481-490.
Niwa, H., Burdon, T., Chambers, I., and Smith, A. G. (1998). Self-renewal of pluripotent embryonic stem cells is mediated via activation of STAT3. Genes Dev. 12, 2048-2060.
Nogueira, M et al (2000). Regulation of Id gene expression during Embryonic stem cell—derived haematopoietic differentiation. Biochem. Biophys Res. Comm. 276, pp 803-812.
Norton, J. D. (2000). ID helix-loop-helix proteins in cell growth, differentiation and tumorigenesis. J. Cell Sci. 113 (Pt 22), 3897-3905.
Ramalho-Santos, M., Yoon, S., Matsuzaki, Y., Mulligan, R. C., and Melton, D. A. (2002). “Stemness”: transcriptional profiling of embryonic and adult stem cells. Science 298, 597-600.
Robertson, E. J. (1987). Embryo-derived stem cell lines. In Teratocarcinomas and embryonic stem cells: a practical approach, E. J. Robertson, ed. (Oxford, IRL Press), pp. 71-112.
Sato, N., Sanjuan, I. M., Heke, M., Uchida, M., Naef, F., and Brivanlou, A. H. (2003). Molecular signature of human embryonic stem cells and its comparison with the mouse. Dev. Biol. 260, 404-413.
Schmitz, J., Weissenbach, M., Haan, S., Heinrich, P. C., and Schaper, F. (2000). SOCS3 exerts its inhibitory function on interleukin-6 signal transduction through the SHP2 recruitment site of gp130. J. Biol. Chem. 275, 12848-12856.
Schofield, R. (1978). The relationship between the spleen colony-forming cell and the hemopoietic stem cell. A hypothesis. Blood Cells 4, 4-7.
Schorpp-Kistner, M., Wang, Z. Q., Angel, P., and Wagner, E. F. (1999). JunB is essential for mammalian placentation. Embo J 18, 934-948.
Shi, Y., and Massague, J. (2003). Mechanisms of TGF-beta signaling from cell membrane to the nucleus. Cell 113, 685-700.
Sirard, C., de la Pompa, J. L., Elia, A., Itie, A., Mirtsos, C., Cheung, A., Hahn, S., Wakeham, A., Schwartz, L., Kern, S. E., et al. (1998). The tumour suppressor gene Smad4/Dpc4 is required for gastrulation and later for anterior development of the mouse embryo. Genes Dev. 12, 107-119.
Smith, A. (2001a). Embryonic Stem Cells. In Stem Cell Biology, D. R. Marshak, Gardner, R. L, Gottlieb, D., ed. (New York, Cold Spring Harbor Laboratory Press), pp. 205-230.
Smith, A. G. (2001 b). Embryo-derived stem cells: of mice and men. Ann. Rev. Cell Dev. Biol. 17, 435-462.
Smith, A. G., Heath, J. K., Donaldson, D. D., Wong, G. G., Moreau, J., Stahl, M., and Rogers, D. (1988). Inhibition of pluripotential embryonic stem cell differentiation by purified polypeptides. Nature 336, 688-690.
Stewart, C. L., Kaspar, P., Brunet, L. J., Bhatt, H., Gadi, I., Kontgen, F., and Abbondanzo, S. J. (1992). Blastocyst implantation depends on maternal expression of leukaemia inhibitory factor. Nature 359, 76-79.
Suda, Y., Suzuki, M., Ikawa, Y., and Aizawa, S. (1987). Mouse embryonic stem cells exhibit indefinite proliferative potential. J. Cell. Physiol. 133, 197-201.
Sun, Y., Nadal-Vicens, M., Misono, S., Lin, M. Z., Zubiaga, A., Hua, X., Fan, G., and Greenberg, M. E. (2001). Neurogenin promotes neurogenesis and inhibits glial differentiation by independent mechanisms. Cell 104, 365-376.
Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.
Tropepe, V., Hitoshi, S., Sirard, C., Mak, T. W., Rossant, J., and van der Kooy, D. (2001). Direct neural fate specification from embryonic stem cells: a primitive mammalian neural stem cell stage acquired through a default mechanism. Neuron 30, 65-78.
Tulina, N., and Matunis, E. (2001). Control of stem cell self-renewal in Drosophila spermatogenesis by JAK-STAT signaling. Science 294, 2546-2549.
von Bubnoff, A., and Cho, K. W. (2001). Intracellular BMP signaling regulation in vertebrates: pathway or network? Dev. Biol. 239, 1-14.
Wadia J. S. et al, Curr. Op. Biotechnology, 2002, 13, pp 52-56.
Wiles, M. V., and Johansson, B. M. (1999). Embryonic stem cell development in a chemically defined medium. Exp Cell Res 247, 241-248.
Williams, R. L., Hilton, D. J., Pease, S., Willson, T. A., Stewart, C. L., Gearing, D. P., Wagner, E. F., Metcalf, D., Nicola, N. A., and Gough, N. M. (1988). Myeloid leukaemia inhibitory factor maintains the developmental potential of embryonic stem cells. Nature 336, 684-687.
Wilson, P. A., and Hemmati-Brivanlou, A. (1995). Induction of epidermis and inhibition of neural fate by Bmp-4. Nature 376, 331-333.
Wilson, S. I., and Edlund, T. (2001). Neural induction: toward a unifying mechanism. Nat. Neurosci. 4 Suppl, 1161-1168.
Winnier, G., Blessing, M., Labosky, P. A., and Hogan, B. L. (1995). Bone morphogenetic protein-4 is required for mesoderm formation and patterning in the mouse. Genes Dev. 9, 2105-2116.
Xie, T., and Spradling, A. C. (1998). decapentaplegic is essential for the maintenance and division of germline stem cells in the Drosophila ovary. Cell 94, 251-260.
Ying, Q. L., Nichols, J., Evans, E. P., and Smith, A. G. (2002). Changing potency by spontaneous fusion. Nature 416, 545-548.
Ying, Q.-L., and Smith, A. G. (2003). Defined conditions for neural commitment and differentiation. In Differentiation of Embryonic Stem Cells, P. Wassarman, and G. Keller, eds. (Elsevier), pp. 327-341.

Ying, Q.-L., Stavridis, M., Griffiths, D., Li, M., and Smith, A. (2003). Conversion of embryonic stem cells to neuroectodermal precursors in adherent monoculture. Nature Biotechnology 21, 183-186.

TABLE
Propagation of single ES cells in serum-free medium with
LIF plus BMP or with LIF alone after Id transfection

| | Parental ES Cells | | Id1 Transfectants | |
|---|---|---|---|---|
| | LIF | LIF + BMP4 | LIF | LIF + BMP4 |
| Number of single cells | 96 | 192 | 192 | 192 |
| picked | | | | |
| Number of colonies formed | 1 | 12 | 19 | 22 |
| at day 8 | | | | |
| Number of colonies | 0 | 10 | 16 | 20 |
| expanded | | | | |

1. A method of promoting self-renewal of pluripotent cells in culture, comprising culturing pluripotent cells in a medium comprising an Id gene product.

2. The method according to claim 1, wherein the medium further comprises an activator of a gp130 downstream signalling pathway.

3. The method according to claim 1, wherein the
medium is free of serum and free of serum extract.

4. The method according to claim 2, wherein the activator of a gp130 downstream signalling pathway is LIF.

5. The method according to claim 1, wherein the pluripotent cells are embryonic stem cells.

6. The method according to claim 5 wherein the embryonic stem cells are mouse cells or human cells.

7. (canceled)

8. The method according to claim 1, further comprising inducing expression of an Id gene.

9. The method according to claim 8, wherein the expression of an Id gene is induced by genetically manipulating a pluripotent cell so that it expresses an Id gene.

10. The method according to claim 8, wherein the expression of an Id gene is induced by introducing into a pluripotent cell a vector comprising an Id gene.

11. The method according to claim 1 wherein the Id gene product is an Id protein.

12. A method of promoting self-renewal of a pluripotent cell in culture in medium that is free of serum and free of serum extract, comprising (1) expressing an Id gene or inducing expression of an Id gene in the cell, or culturing the cell in medium containing an Id protein, and (2) activating gp130 downstream signalling.

13. The method according to claim 12, comprising expressing an Id gene episomally in the cell.

14. The method according to claim 13 comprising expressing an id gene from an episomal vector comprising an inducible promoter.

15. The method according to claim 12, comprising activating gp130 downstream signalling by culturing the cell in medium comprising a cytokine acting through gp130.

16. The method according to claim 15 wherein the cytokine is selected from the group consisting of LIF, CNTF, Cardiotrophin, Oncostatin M and a combination of IL-6 plus sIL-6 receptor.

17. A method of promoting self-renewal of a pluripotent cell in culture, comprising culturing a pluripotent cell in a medium comprising:
(a) a direct activator or effector of Id gene expression and/or Id protein activity, other than one acting through a receptor of the TGF-β superfamily; and
(b) an activator of a gp130 downstream signalling pathway, wherein the medium is free of serum and free of serum extract.

18. A method of culture of ES cells to promote ES cell self renewal in medium that is free of serum and free of serum extract, comprising maintaining the ES cells in medium comprising:
(a) an Id protein or a direct activator or effector of Id gene expression and/or Id protein activity, other than one acting through a receptor of the TGF-β superfamily; and
(b) an activator of a gp130 downstream signalling pathway.

19. A method of culture of ES cells, comprising the steps of:
(a) maintaining the ES cells in a pluripotent state in culture, optionally on feeders feeder cells, in the presence of a cytokine acting through gp130 and serum or an extract of serum;
(b) passaging the ES cells at least once;
(c) withdrawing the serum or the serum extract from the medium and withdrawing the feeder cells if present, so that the medium is free of feeder cells, serum and serum extract; and
(d) subsequently maintaining ES cells in a pluripotent state by culturing the cells in a medium comprising:
(i) a direct activator or effector of Id gene expression and/or Id protein activity, other than one acting through the receptor of the TGF-β superfamily; and
(ii) an activator of a gp130 downstream signalling pathway.

20. A method of obtaining a transfected population of ES cells, comprising the steps of:
(a) transfecting ES cells with a construct encoding a selectable marker operably linked to a promoter that expresses the selectable marker preferentially in an ES cell:
(b) plating the ES cells;
(c) culturing the ES cells in the presence of
(i) a direct activator or effector of Id gene expression and/or Id protein activity, other than one activator acting through a receptor of the TGF-β superfamily; and
(ii) an activator of a gp130 downstream signalling pathway; and
(d) selecting for cells that express the selectable marker.

21. A method of culture of ES cells in medium that is free of serum and free of serum extract, comprising transferring an individual ES cell to a culture vessel and culturing the ES cell in the presence of
(a) a direct activator or effector of Id gene expression and/or Id protein activity, other than one acting through a receptor of the TGF-β superfamily; and
(b) an activator of a gp130 downstream signalling pathway, so as to obtain a clonal population of ES cells, all of which are progeny of a single ES cell.

22. A medium for self-renewal of ES cells, comprising:
(a) basal medium;
(b) a direct activator or effector of Id gene expression and/or Id protein activity, other than one acting through a receptor of the TGF-β superfamily;
(c) an activator of gp130 downstream signalling pathways; and
(d) an iron transporter,
wherein the medium is free of serum or serum extract.

23. A method of promoting self-renewal of pluripotent cells in culture, comprising culturing pluripotent cells in a medium comprising an agent that increases Id protein activity, wherein the medium free of serum and free of serum extract.

24. The method according to claim 23 wherein the agent increases the amount of Id protein in the cell.

25. The method according to claim 23 wherein the agent comprises a composition comprising an Id protein and a translocation domain.

26. The method according to claim 25, wherein the composition comprises a fusion protein of the Id protein and the translocation domain.

27. The method according to claim 25, wherein the translocation domain comprises TAT, VP22 or a penetratin.

28. A method of obtaining a pluripotent cell in medium that is free of serum and free of serum extract, comprising
expressing an Id gene or inducing expression of an Id gene in a cell, or culturing a cell in medium containing an Id protein, and activating gp130 downstream signalling in the cell, wherein the cell is obtained from somatic cells or tissue of a fetus or adult.

29. The method according to claim 28, wherein the pluripotent cell is characterised by being positive for Rex1, Oct4 and nanog.

30. A cell obtained by the method of claim 28.

31. A method of promoting self-renewal of pluripotent cells in culture, comprising culturing pluripotent cells in a medium comprising:
(a) an agent that increases Id gene expression or activity; and
(b) an activator of a gp130 downstream signalling pathway, wherein the medium is free of serum and free of serum extract.

32. The method according to claim 31, wherein the activator of a gp130 downstream signalling pathway is LIF.

33. The method according to claim 31, wherein the pluripotent stem cells are embryonic stem cells.

34. The method according to claim 33, wherein the embryonic stem cells are mouse cells or human cells.

35. The method according to claim 31, wherein the agent (i) is selected from the group consisting of fibronectin, agonists of the fibronectin receptor, activators of integrin signalling, nanog, and homologues thereof that induce Id gene expression or Id protein activity.

36. The method according to claim 31, further comprising inducing expression of an Id gene.

37. The method according to claim 36, wherein the expression of an Id gene is induced by genetically manipulating a pluripotent cell so that it expresses an Id gene.

38. The method according to claim 36, wherein the expression of an Id gene is induced by introducing into a pluripotent cell a vector comprising an Id gene.


SEQUENCE LISTING (9 sequences)

SEQ ID NO: 1 - protein, 119 aa, Mus sp.
MKALSPVRGCYEAVCCLSERSLAIARGRGKSPSTEEPLSLLDDMNHCYSRLRELVPGVPRGTQLSQVEILQRVIDYILDLQVVLAEPAPGPPDGPHLPIQTAELTPELVISKDKRSFCH

SEQ ID NO: 2 - protein, 119 aa, Rattus sp.
MKALSPVRGCYEAVCCLSERSLAIARGRGKSPSAEEPLSLLDDMNHCYSRLRELVPGVPRGTQLSQVEILQRVIDYILDLQVVLAEPAPGPPDGPHLPIQTAELTPELVISKDKRSFCH

SEQ ID NO: 3 - protein, 119 aa, Canis sp.
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELAPELVISNDKRSFCH

SEQ ID NO: 4 - protein, 119 aa, Homo sapiens; identical to SEQ ID NO: 3

SEQ ID NO: 5 - protein, 11 aa, Human immunodeficiency virus
YGRKKRRQRRR

SEQ ID NO: 6 - protein, 16 aa, Antennapedia
RQIKIWFQNRRMKWKK

SEQ ID NO: 7 - protein, 130 aa, Artificial Sequence
  synthetic
YGRKKRRQRRRMKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELAPELVISNDKRSFCH

SEQ ID NO: 8 - protein, 135 aa, Artificial Sequence
  synthetic
RQIKIWFQNRRMKWKKMKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELAPELVISNDKRSFCH

SEQ ID NO: 9 - protein, 135 aa, Artificial sequence
  synthetic
MKALSPVRGCYEAVCCLSERSLAIARGRGKSPSTEEPLSLLDDMNHCYSRLRELVPGVPRGTQLSQVEILQRVIDYILDLQVVLAEPAPGPPDGPHLPIQTAELTPELVISKDKRSFCHRQIKIWFQNRRMKWKK